(19) United States
(12) Patent Application Publication
(10) Pub. No.: US 2014/0378726 A1
(43) Pub. Date: Dec. 25, 2014

(54) CATALYTIC CONVERSION PROCESSES USING IONIC LIQUIDS

(71) Applicant: UOP LLC, DES PLAINES, IL (US)

(72) Inventors: BIPIN V. VORA, NAPERVILLE, IL (US); BING SUN, SOUTH BARRINGTON, IL (US)

(21) Appl. No.: 13/923,145

(22) Filed: Jun. 20, 2013

(51) Int. Cl. C07C 5/327

(52) U.S. Cl. CPC C07C 5/327
USPC 585/327

(57) ABSTRACT

A process for making isoprene from isobutane is described. The process allows control of the isobutene concentration entering the isoprene reaction zone to be at least about 40% consistently. The process also allows control of the oxygenate removal zone.

### BACKGROUND OF THE INVENTION

Olefinic hydrocarbons are feedstocks for a variety of commercially important reactions to yield fuels, polymers, oxygenates and other chemical products. Butenes are among the most useful of the olefinic hydrocarbons having more than one isomer. A high-octane gasoline component is produced from a mixture of butenes in many petroleum refineries, principally by alkylation with isobutane; 2-butenes (cis- and trans) generally are the most desirable isomers for this application. Secondary butyl alcohol and methylethyl ketone, as well as butadiene, are other important derivatives of butenes. Demand for 1-butene has been growing rapidly, based on its use as a co-monomer for linear low density polyethylene and as a monomer in polybutene production. Isobutene finds application in such products as methyl tert-butyl ether (MTBE), methyl methacrylate, polyisobutene and butyl rubber.

The dehydrogenation of hydrocarbons is well described in the prior art, with both acyclic and aromatic hydrocarbons being thereby converted to the corresponding less saturated products. A typical prior art process flow comprises the admixture of the feed hydrocarbon with hydrogen and the heating of the feed stream through indirect heat exchange with the dehydrogenation zone effluent stream. After being heated in a heat exchanger, the feed stream is further heated by passage through a heater which is typically a fired heater or furnace. The feed stream is then contacted with a bed of dehydrogenation catalyst, which may be either a fixed, moving or fluidized bed of catalyst. The dehydrogenation reaction is very endothermic. To counteract this cooling effect of the reaction, heat may be supplied to the bed of dehydrogenation catalyst in various ways, including indirect heat exchange with circulating high temperature fluids, rapid turnover of catalyst in a fluidized bed system, or a multistage reaction zone with interstage heaters.

The effluent stream from the dehydrogenation reaction zone is cooled sufficiently to cause a partial condensation of the effluent stream. The partial condensation facilitates the easy separation of the bulk of the hydrogen from the other components of the effluent stream, with a portion of the hydrogen being removed as a net product gas and a second portion normally being recycled to the dehydrogenation reaction zone. The remaining mixture of saturated and unsaturated hydrocarbons and by-products is sent to the appropriate products recovery facilities, which will typically comprise a first stripping column which removes light ends having boiling points below that of the desired product and a second fractionation column which separates the remaining hydrocarbons into product and recycle streams.

The product streams can then be further processed into other products using various conversion processes depending on the desired product.

For example, in a C₄ complex, the separator liquid from the dehydrogenation zone is sent, either directly or after a depropanizer as a depropanizer bottoms stream, to an isobutene-consuming process, such as an isoprene reaction zone. Approximately 80% to 90% of the isobutene is converted to isoprene with the addition of formaldehyde.

The raffinate C4, unconverted isobutene, and some normal butanes can then be processed in an etherification reaction zone to convert the remaining isobutene into methyl tert-butyl ether (MTBE). The raffinate stream from the etherification reaction zone is then processed in an oxygenate removal zone and a saturation zone. The stream from the saturation zone can be sent to a deisobutanizer column to reject the normal butanes, or recycled directly to the dehydrogenation reaction zone, depending on the normal C₄ content.

Often the downstream isobutene consuming process unit has strict requirements for the feed coming to the unit, for example, maximum or minimum concentrations of components such as C₃, normal C₄, or butadiene content, etc., because of the catalyst being used and the like.

For example, a process for isoprene production requires a minimum concentration of about 45 wt % isobutene, a maximum of about 1-2% C₃, and a very low, less than 10 ppm butadiene content.

Therefore, there is a need for a process which efficiently controls of the production of isoprene.

### SUMMARY OF THE INVENTION

One aspect of the invention is a process for making isoprene. The process includes dehydrogenating a feed comprising isobutane in a dehydrogenation zone under dehydrogenation conditions to form a mixture comprising isobutene, and isobutane and separating the mixture into a liquid effluent stream and a gaseous effluent stream. The liquid effluent stream is separated in a depropanizer into an overhead vapor stream comprising C₃₋ hydrocarbons and a depropanizer stream comprising the isobutene, and isobutane. The depropanizer stream is introduced into an isoprene reaction zone, the depropanizer stream having a concentration of isobutene of at least about 40%. The isobutene is reacted with formaldehyde in the isoprene reaction zone to form a mixture comprising isoprene, isobutene, and isobutane, wherein the conversion of isobutene to isoprene is less than 99%. The mixture from the isoprene reaction zone is separated into a product stream comprising isoprene and an isoprene effluent stream comprising isobutene, and isobutane. The product stream comprising isoprene is recovered. The isoprene effluent stream is introduced to an etherification zone. The isobutene is reacted with methanol in the etherification reaction zone to form a mixture comprising methyl tert-butyl ether, isobutene, and isobutane. The mixture from the etherification zone is separated into a product stream comprising methyl tert-butyl ether and an effluent stream comprising isobutene, and isobutane. Oxygenated compounds from the effluent stream are removed in an oxygenate removal zone. Isobutene from the effluent stream is reacted with hydrogen in a saturation zone to form an isobutane stream comprising isobutane.

### BRIEF DESCRIPTION OF THE DRAWINGS

FIG. 1 is a simplified flow scheme illustrating one embodiment of an isoprene manufacturing process.

FIG. 2 is a simplified flow scheme illustrating another embodiment of an isoprene manufacturing process.

FIG. 3 is a simplified flow scheme illustrating another embodiment of an isoprene manufacturing process.

FIG. 4 is a simplified flow scheme illustrating still another embodiment of an isoprene manufacturing process.

### DETAILED DESCRIPTION OF THE INVENTION

The dehydrogenation reaction is a reversible, equilibrium limited reaction. Depending on the temperature and pressure at which the reaction takes place, there is a maximum concentration that can be formed, and it is not possible to go beyond that maximum. In order to obtain greater than about 50% conversion of isobutane to isobutene at atmospheric pressure, a higher temperature (e.g., about 640° C. to 650° C.) is required. However, this increased temperature is undesirable. As a result, the conversion limit is 50% at atmospheric pressure and 620° C., generally in the range of about 45% to about 50%. Moreover, the catalyst can become less effective with time as coke builds up on it.

On the other hand, it is desirable that the concentration of isobutene entering the isoprene reactor be at least about 40%, or at least about 45%, or at least about 47%, and desirably at least about 50%.

The present invention allows efficient control of the process to obtain the desired concentrations without having to increase the temperature of the dehydrogenation reaction zone to an undesirable level. It involves managing the isobutene concentration entering the isoprene reaction zone to be at least about 40% consistently and preferably above 45%, even if the dehydrogenation catalyst becomes less effective. It also involves managing the oxygenate removal zone.

The process for the production isoprene from an isobutane feedstock, typically includes a dehydrogenation reaction zone, a depropanizer column, an isoprene reaction zone, an etherification reaction zone, an oxygenate removal zone, and a saturation zone. It can include an optional sulfur removal zone, and gas separation zone. There can be additional equipment, as is known in the art.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones. The dehydrogenation reaction zone is typically operated at a temperature of in the range from 550° C. and 700° C. To avoid unsafe vacuum pressure conditions, the reactor zone is typically operated above atmospheric pressure, e.g., between about 7 kPa (g) to about 700 kPa (g), or about 7 kPa (g) to about 350 kPa (g), or about 20 kPa (g) to about 205 kPa (g). The reaction is operated under a hydrogen partial pressure, and the hydrogen to hydrocarbon mole feed ratio at the reactor inlets is less than 0.8.

In some embodiments, the bottoms stream from the depropanizer, which contains C₄ and C₅₋ hydrocarbons, is sent to the isoprene reaction zone. In some embodiments, the C₅₊ hydrocarbons and any DME formed in the etherification reaction zone are removed in a raffinate stripper column. In other embodiments, the C₅₊ hydrocarbons and any DME formed in the etherification reaction zone are sent back to the depropanizer column where the DME is removed overhead with the C₃₋ hydrocarbons. The C₅₊ hydrocarbons will exit in the bottoms stream.

In one embodiment, a liquid side cut stream is taken from the depropanizer at about 3 to 7 trays from the top. The liquid side cut is small, typically about 2 to 10% of the feed stream, and is mainly isobutane. As s result of removing the isobutane, the isobutene content in the bottoms stream is increased. The side cut stream bypasses the isoprene reaction zone, and goes to the etherification reaction zone. Therefore, it is fully recovered, and can be recycled back to the dehydrogenation reaction zone. In this case, quantity of the isobutane side cut is determined by the desired isobutene concentration in the bottoms stream going to the isoprene reaction zone.

In other embodiments, a bottoms side cut of C₄ is taken about 5 trays from the bottoms and sent to the isoprene reaction zone. In this case, the bottom stream is primarily 2-butene, n-butane and C₅₊ hydrocarbons. In this mode, by removing 2-butenes, small amount of n-butane and C₅₊ material, the concentration of isobutene in the sidecut stream to the isoprene reaction zone is increased.

In some embodiments, isobutane from the saturation zone is used to desorb oxygenate compounds from the adsorbent of the oxygenate removal zone. In some embodiments, the isobutane with the oxygenate compounds are burned to recover their heating value. In other embodiments, the isobutane with the oxygenate compounds are recycled to the depropanizer.

In other embodiments, the C₃₋ hydrocarbons are used to desorb oxygenate compounds from the adsorbent of the oxygenate removal zone. The C₃₋ hydrocarbons/DME with the oxygenate compounds can be burned to recover its heating value.

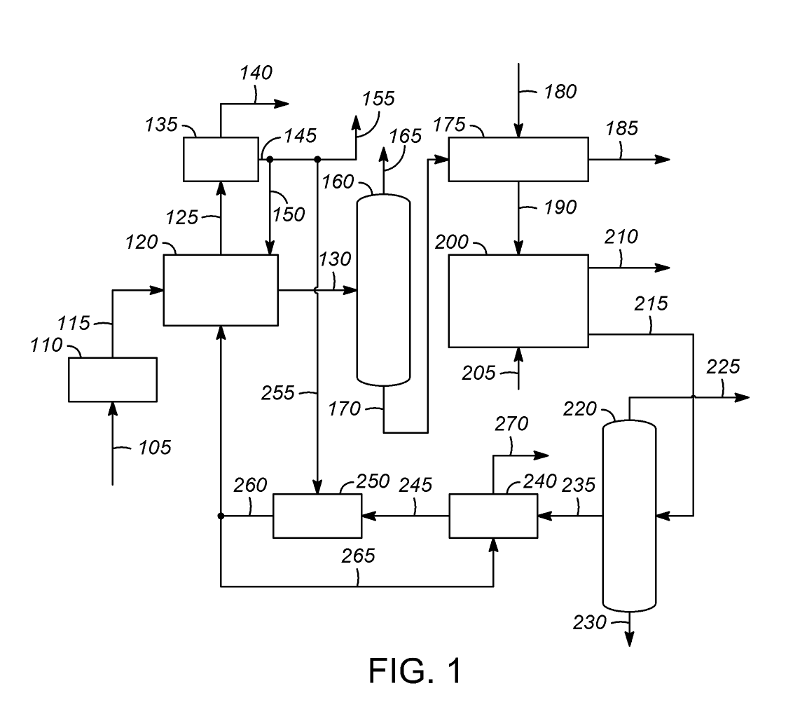

FIG. 1 illustrates one embodiment of an isoprene manufacturing process according to the present invention. Fresh feed 105 comprises isobutane. The fresh feed will typically contain at least about 90% isobutane, with the remainder being normal butane and a small amount of propane. The feed will desirably contain at least about 95% isobutane, or at least about 97%.

The feed 105 can be obtained from a deisobutanizer column (not shown) which separates isobutane as an overhead stream and normal butane as a bottoms stream. The overhead stream will typically be about 95 to about 99% isobutane with the balance being normal butane. If there is propane in the initial C₄ feed, the propane will remain in the overhead isobutane stream.

If the sulfur level is exceeds acceptable levels for the dehydrogenation catalyst, the fresh feed 105 can be sent to a desulfurization zone 110 for removal of sulfur. If the sulfur levels are acceptable, the desulfurization zone is not needed.

The desulfurized feed 115 flows to a dehydrogenation reaction zone 120 where the isobutane is dehydrogenated into isobutene. The conversion of isobutane to isobutene is about 50% (the conversion typically rages from about 45% to about 55%), with a selectivity of over 90%. The conversion of normal butane to normal butene is also about 50%, with a selectivity of about 80% to 85% because more cracking occurs with normal butane. The normal butane is dehydrogenated into butene-1, cis-butene-2, or trans-butene-2. Small amounts of butadiene may also be formed in the dehydrogenation reaction zone 120. The reaction mixture will also include unreacted isobutane and normal butane. The reaction mixture is separated into a gaseous effluent stream 125 and a liquid effluent stream 130.

The gaseous affluent stream 125, containing light hydrocarbons, methane, some ethane, ethylene and primarily hydrogen, is sent to a gas separation zone 135 where it is separated into a methane rich stream 140 and a hydrogen stream 145. One example of a suitable gas separation zone is a pressure swing adsorption (PSA) unit. Depending on the separation process, the hydrogen stream 145 can be a highly pure stream, e.g., 99+% pure hydrogen.

The methane rich stream 140 can be used for fuel if desired.

In some embodiments, a portion 150 of the hydrogen stream 145 is sent back to the dehydrogenation reaction zone 120. When a platinum catalyst is used, the presence of some hydrogen at the inlet with the feed has been found to reduce coking and improve catalyst stability.

In some embodiments, another portion 155 of the hydrogen stream 145 is recovered for use in other processes, such as olefin saturation zone discussed later or other processes (not shown).

The liquid effluent stream 130 from the dehydrogenation reaction zone 120 containing the isobutene, normal butene, isobutane, and normal butane is sent to the depropanizer column 160. The liquid effluent stream 130 is separated into an overhead stream 165 containing C₃₋ hydrocarbons and a depropanizer bottom stream 170 including the isobutene, normal butene, isobutane, and normal butane. The depropanizer bottom stream also includes any C₅₊ hydrocarbons that were present in the feed to the dehydrogenation or formed in the dehydrogenation reaction zone.

The depropanizer bottom stream 170 has a concentration of isobutene of at least about 40%, or at least about 45%, or atl east about 47%, or at least about 50%.

The depropanizer bottom stream 170 is sent to the isoprene reaction zone 175 where the isobutene is reacted with formaldehyde 180 to form isoprene. The reaction mixture includes isoprene, unreacted isobutene, and isobutane (as well as any normal butenes, normal butane, etc.).

The conversion of isobutene to isoprene is desirably less than about 99% in order to limit reaction byproducts. The conversion is desirably less than about 95%, or less than about 90%, or less than about 85%, or less than about 80%, or in the range of about 80% to about 90%.

The reaction mixture is separated into a product stream 185 comprising isoprene, which is recovered, and an isoprene effluent stream 190 comprising unreacted isobutene and isobutane (as well as any normal butenes, normal butane, etc.).

The isoprene effluent stream 190 is sent to an etherification reaction zone 200 where isobutene is reacted with methanol 205 to form methyl tert-butyl ether (MTBE). The reaction mixture is separated into a product stream 210 comprising MTBE and a raffinate stream 215 comprising

isobutane (and any normal butane/butenes). The raffinate stream may contain a small amount of dimethylether (DME) if it is formed in the etherification reaction zone 200.

The raffinate stream 215 is sent to a raffinate stripper column 220 for separation into an overhead stream 225 containing any DME formed, a bottoms stream 230 comprising the C₅₊ hydrocarbons, and a side cut stream 235 containing the isobutane (and any normal butane/butenes). The DME stream 225 is removed and could be used as fuel. The bottoms stream 230 is removed and could be used in gasoline blending or as a fuel.

The side cut stream 235 is sent to the oxygenate removal zone 240. Oxygenate compounds are used and/or can be formed at various points in the process. For example, there could be unreacted formaldehyde from the isoprene reaction, unreacted methanol from the etherification reaction, and as mentioned earlier, DME can form in the etherification zone. These oxygenate compounds need to be removed from the process stream as they are harmful to platinum containing dehydrogenation catalysts. The oxygenate removal zone 240 typically is an adsorption unit. The oxygenate compounds are removed using an adsorbent such as molecular sieves.

The stream 245 containing isobutane from the oxygenate removal zone 240 is sent to the saturation zone 250 where the normal butenes and any remaining unconverted isobutene are hydrogenated with hydrogen. In some embodiments, the hydrogen is a portion 255 of the hydrogen stream 145 from the gas separation zone 135.

The effluent 260 from the saturation zone 250 is primarily isobutane which is sent back to the dehydrogenation zone 120.

In some embodiments, a portion 265 of the effluent 260 can be recycled back to the oxygenate removal zone 240 to desorb the oxygenate compounds from the adsorbent. The stream 270 of isobutane with desorbed oxygenate compounds can be burned for fuel to recover its heating value, if desired. Alternatively, it can be recycled back to the etherification reaction zone 200 to recover the isobutane. The absorbed oxygenates will be rejected through the raffinate stripping column 220.

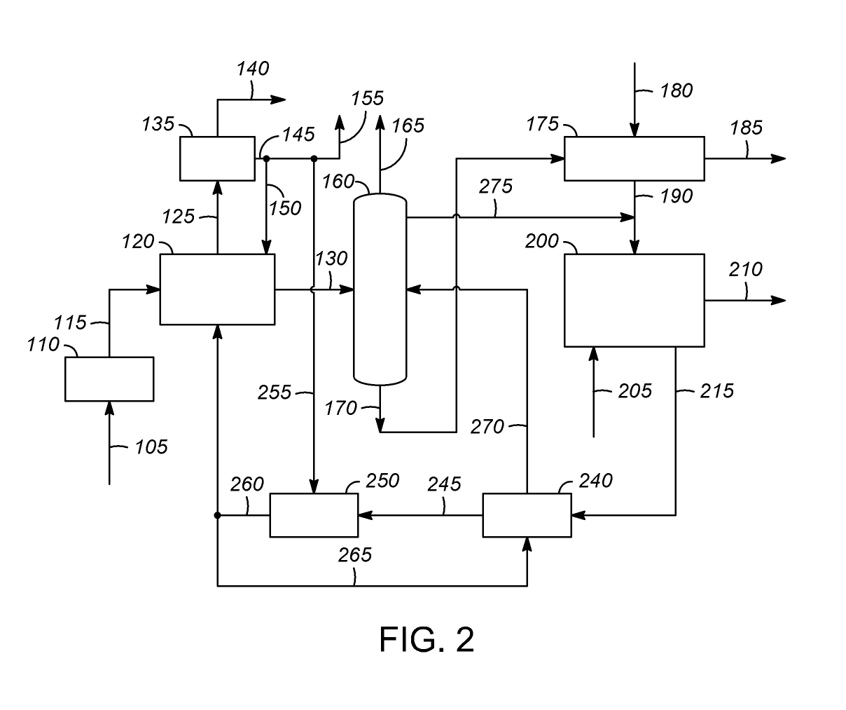

FIG. 2 illustrates another embodiment of an isoprene manufacturing process according to the present invention. Fresh isobutane feed 105 can be sent to the desulfurization zone 110 for removal of sulfur, if needed.

The desulfurized feed 115 flows to the dehydrogenation reaction zone 120 where the isobutane is dehydrogenated into isobutene. The reaction mixture is separated into a gaseous effluent stream 125 and a liquid effluent stream 130.

The gaseous affluent stream 125 contains methane, some ethane, ethylene and primarily hydrogen, which are separated into a methane rich stream 140 and a hydrogen stream 145 in gas separation zone 135.

In some embodiments, a portion 150 of the hydrogen 145 is sent back to the dehydrogenation reaction zone 120. In some embodiments, another portion 155 of the hydrogen 145 is recovered.

The liquid effluent stream 130 from the dehydrogenation reaction zone 120 containing the isobutene, normal butene, isobutane, and normal butane is sent to the depropanizer column 160. The liquid effluent stream 130 is separated into an overhead stream 165 containing C₃₋ hydrocarbons and a depropanizer bottom stream 170 including the isobutene, normal butene, isobutane, and normal butane. The depropanizer bottom stream also includes any C₅₊ hydrocarbons that were in the feed to the dehydrogenation or were formed in the dehydrogenation reaction zone.

TABLE 1

| Component | Normal BP, ° C. |
|---|---|
| Propylene | −47.4 |
| Propane | −42.1 |
| DME | −25 |
| Isobutane | −11.7 |
| Isobutene | −6.8 |
| Butene-1 | −6.3 |
| 1,3 Butadiene | −4.41 |
| n-Butane | −0.6 |
| Tr-butene-2 | 0.9 |
| Cis Butene-2 | 3.7 |
| Isopentane | 27.8 |
| Ref: Hand book of Chemistry | |
| and Physics, 62^{nd} Ed. 1981-82 | |

Distillation columns are designed to separate lighter boiling components from higher boiling components. From Table 1 above, it seen that DME, propane, and propylene are significantly lighter boiling components and, if present, will be removed as an overhead steam. Isobutane is a valuable feed component for the production of isobutene which is subsequently converted to isoprene, and therefore, maximum recovery of isobutane is desirable. By proper design of number of trays and reflux rate, one can pick an upper sidecut tray location that maximizes concentration of isobutane with minimal isobutene.

A liquid upper sidecut stream 275 is taken at about 3 to 10 trays from the top of the depropanizer column 160. The liquid sidecut stream 275 is mostly isobutane. The quantity of this stream controlled by the desired concentration of isobutene in the bottoms stream for the isoprene reaction zone. Typically, this is a small stream, e.g., less than about 5 wt % of the bottoms stream, or less than about 4 wt %, or less than about 3 wt %, or about 2 to about 5 wt %. As a result of the removal of the isobutane, the level of isobutene in the depropanizer bottom stream 170 becomes more than 45 wt %. The upper sidecut stream 275 bypasses the isoprene reaction zone 175 and is sent to the etherification zone 200. Thus, it is fully recovered and sent back to the dehydrogenation reaction zone 120.

The depropanizer bottom stream 170 is sent to the isoprene reaction zone 175 where the isobutene is reacted with formaldehyde 180 to form isoprene. The reaction mixture is separated into a product stream 185 comprising isoprene, which is recovered, and an isoprene effluent stream 190 comprising unreacted isobutene and isobutane (as well as any normal butenes, normal butane, etc.).

The isoprene effluent stream 190 is sent to the etherification reaction zone 200 where isobutene is reacted with methanol 205 to form MTBE. The reaction mixture is separated into a product stream 210 comprising MTBE and a raffinate stream 215 comprising isobutane (and any normal butane/butenes).

The raffinate stream 215 is sent to the oxygenate removal zone 240. The stream 245 containing any unreacted isobutene, isobutane, and normal butane/butenes from the oxygenate removal zone 240 is sent to the saturation zone 250 where the isobutene and normal butenes are hydrogenated with hydrogen. The effluent 260 from the saturation zone 250 is primarily isobutane which is sent back to the dehydrogenation zone 120.

A portion 265 of the effluent 260 can be recycled back to the oxygenate removal zone 240 to desorb the oxygenate compounds from the adsorbent. The stream 270 of isobutane with desorbed oxygenate compounds can be sent to the depropanizer column 160. DME being a lighter boiling component, is distilled overhead with the C₃— hydrocarbons. The isobutane is recovered in the upper sidecut stream or in the bottoms stream. This provides a significant improvement over the previous flow scheme, where the spent adsorbent regenerant was burned as fuel.

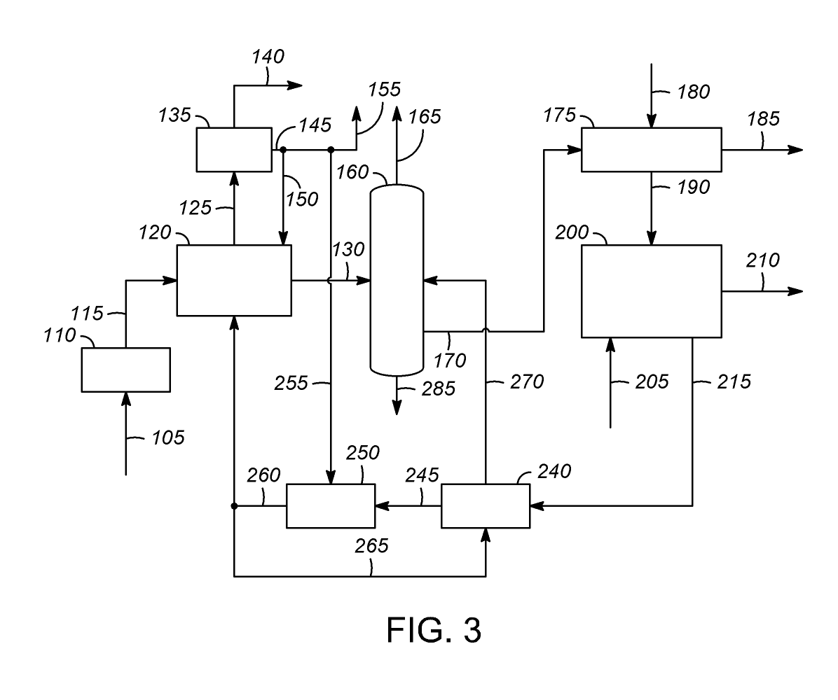

FIG. 3 illustrates another embodiment of an isoprene manufacturing process according to the present invention. Fresh isobutane feed 105 can be sent to the desulfurization zone 110 for removal of sulfur, if needed.

The desulfurized feed 115 flows to the dehydrogenation reaction zone 120 where the isobutane is dehydrogenated into isobutene. The reaction mixture is separated into a gaseous effluent stream 125 and a liquid effluent stream 130.

The gaseous affluent stream 125 contains methane and hydrogen, which are separated into a methane stream 140 and a hydrogen stream 145 in gas separation zone 135.

In some embodiments, a portion 150 of the hydrogen 145 is sent back to the dehydrogenation reaction zone 120. In some embodiments, another portion 155 of the hydrogen 145 is recovered.

The liquid effluent stream 130 from the dehydrogenation reaction zone 120 containing the isobutene, normal butene, isobutane, and normal butane is sent to the depropanizer column 160. The liquid effluent stream 130 is separated into an overhead stream 165 containing C₃₋ hydrocarbons, a depropanizer lower sidecut stream 170 including the isobutene, normal butene, isobutane, and normal butane, and a bottoms stream 280 containing normal butane and any C₅₊ hydrocarbons. As shown in the table above, normal butane, 2-butenes, and isopentane are significantly higher boiling components compared to the isobutene and can be separated as a heavy bottoms stream without losing any significant isobutane. In this case, the depropanizer lower sidecut stream 170 is taken out about 5 trays from the bottom. The bottoms stream 280 is a small stream, typically less than 5% of the feed stream, but it is rich in normal butane, 2-butene and C₅₊. By removing these components as a bottoms heavy stream, the concentration of isobutene in the lower side cut increases to the desired 45% or greater.

The depropanizer lower sidecut stream 170 is sent to the isoprene reaction zone 175 where the isobutene is reacted with formaldehyde 180 to form isoprene. The reaction mixture is separated into a product stream 185 comprising isoprene, which is recovered, and an isoprene effluent stream 190 comprising unreacted isobutene and isobutane (as well as any normal butenes, normal butane, etc.).

The isoprene effluent stream 190 is sent to the etherification reaction zone 200 where isobutene is reacted with methanol 205 to form MTBE. The reaction mixture is separated into a product stream 210 comprising MTBE and a raffinate stream 215 comprising unreacted isobutene, and isobutane.

The effluent stream 215 is sent to the oxygenate removal zone 240. The stream 245 containing isobutene, isobutane (and any normal butane/butenes) from the oxygenate removal zone 240 is sent to the saturation zone 250 where the isobutene and normal butenes are hydrogenated with hydrogen. The effluent 260 from the saturation zone 250 is isobutane which is sent back to the dehydrogenation zone 120.

A portion 265 of the effluent 260 can be recycled back to the oxygenate removal zone 240 to desorb the oxygenate compounds from the adsorbent. The stream 270 of isobutane with desorbed oxygenate compounds can be sent to the depropanizer column 160.

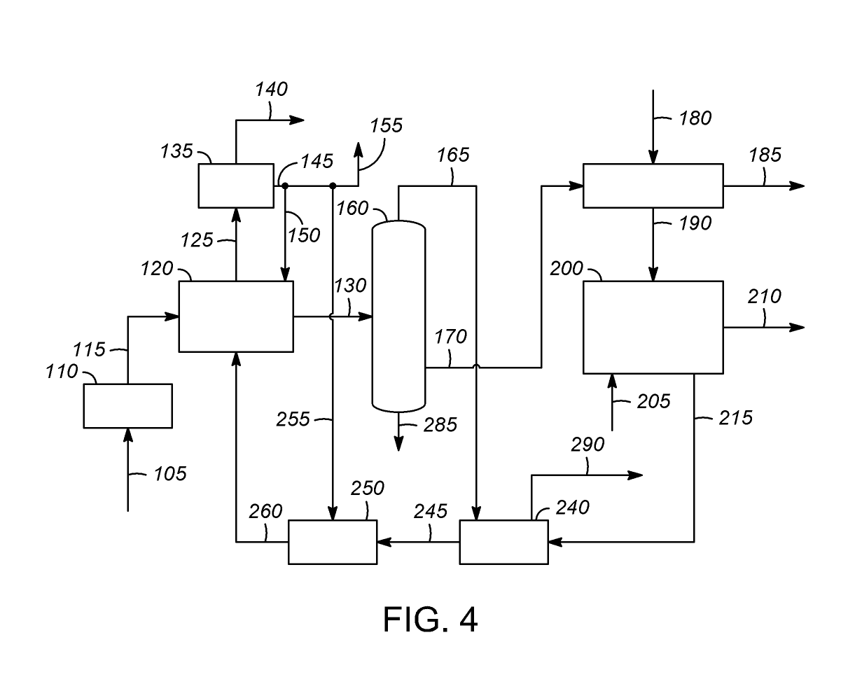

FIG. 4 illustrates still another embodiment of an isoprene manufacturing process according to the present invention. Fresh isobutane feed 105 can be sent to the desulfurization zone 110 for removal of sulfur, if needed.

The desulfurized feed 115 flows to the dehydrogenation reaction zone 120 where the isobutane is dehydrogenated into isobutene. The reaction mixture is separated into a gaseous effluent stream 125 and a liquid effluent stream 130.

The gaseous affluent stream 125 contains methane and hydrogen, which are separated into a methane stream 140 and a hydrogen stream 145 in gas separation zone 135.

In some embodiments, a portion 150 of the hydrogen 145 is sent back to the dehydrogenation reaction zone 120. In some embodiments, another portion 155 of the hydrogen 145 is recovered.

The liquid effluent stream 130 from the dehydrogenation reaction zone 120 containing the isobutene, normal butene, isobutane, and normal butane is sent to the depropanizer column 160. The liquid effluent stream 130 is separated into an overhead stream 165 containing C₃₋ hydrocarbons, a depropanizer side cut stream 170 including the isobutene, normal butene, isobutane, and normal butane, and a bottoms stream 280 containing normal butane and any C₅₊ hydrocarbons. In this case, the depropanizer side cut stream 170 is taken out about 5 trays from the bottom. A significant part of the n-butane, 2-butenes and C₅₊ material contained in the feed to the depropanizer is removed in the bottoms stream 280.

The depropanizer side cut stream 170 is sent to the isoprene reaction zone 175 where the isobutene is reacted with formaldehyde 180 to form isoprene. The reaction mixture is separated into a product stream 185 comprising isoprene, which is recovered, and an isoprene effluent stream 190 comprising isobutene and isobutane (as well as any normal butenes, normal butane, etc.).

The isoprene effluent stream 190 is sent to the etherification reaction zone 200 where it is reacted with methanol 205 to form MTBE. The reaction mixture is separated into a product stream 210 comprising MTBE and a raffinate stream 215 comprising isobutene, and isobutane.

The raffinate stream 215 is sent to the oxygenate removal zone 240. The stream 245 containing unreacted isobutene, isobutane and normal butane/butenes from the oxygenate removal zone 240 is sent to the saturation zone 250 where the isobutene and normal butenes are hydrogenated with hydrogen. The effluent 260 from the saturation zone 250 is isobutane with some normal butane which is sent back to the dehydrogenation reaction zone 120.

The overhead stream 165 is sent to the oxygenate removal zone 240 to desorb the oxygenate compounds from the adsorbent. The stream 290 of propane with desorbed oxygenate compounds can be burned for fuel to recover its heating value, if desired.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for making isoprene comprising:
dehydrogenating a feed comprising isobutane in a dehydrogenation zone under dehydrogenation conditions to form a mixture comprising isobutene, and isobutane and separating the mixture into a liquid effluent stream and a gaseous effluent stream;
separating the liquid effluent stream in a depropanizer into an overhead vapor stream comprising C₃₋ hydrocarbons and a depropanizer stream comprising the isobutene, and isobutane;
introducing the depropanizer stream into an isoprene reaction zone, the depropanizer stream having a concentration of isobutene of at least about 40%;
reacting the isobutene with formaldehyde in the isoprene reaction zone to form a mixture comprising isoprene, isobutene, and isobutane, wherein a conversion of isobutene to isoprene is less than 99%;
separating the mixture from the isoprene reaction zone into a product stream comprising isoprene and an isoprene effluent stream comprising isobutene, and isobutane;
recovering the product stream comprising isoprene;
introducing the isoprene effluent stream to an etherification zone;
reacting the isobutene with methanol in the etherification reaction zone to form a mixture comprising methyl tert-butyl ether, isobutene, and isobutane;
separating the mixture from the etherification zone into a product stream comprising methyl tert-butyl ether and an effluent stream comprising isobutene, and isobutane;
removing oxygenated compounds from the effluent stream in an oxygenate removal zone forming an effluent stream with a reduced level of oxygenated compounds; and
reacting isobutene from the effluent stream with the reduced level of oxygenated compounds with hydrogen in a saturation zone to form an isobutane stream comprising isobutane.

2. The process of claim 1 further comprising recycling a portion of the isobutane stream from the saturation zone to the oxygenate removal zone.

3. The process of claim 1 wherein the effluent stream from the etherification zone further comprises dimethyl ether, and the depropanizer stream further comprises C₅₊ hydrocarbons and further comprising:
separating the effluent stream from the etherification zone into a stream comprising dimethyl ether, a bottoms stream comprising the C₅₊ hydrocarbons, and a side cut stream comprising the isobutene, and isobutane; and
wherein the side cut stream is sent to the oxygenate removal zone.

4. The process of claim 1 wherein the oxygenate removal zone includes an adsorbent, and further comprising:
recycling a portion of the isobutane stream to the oxygenate removal zone; and
desorbing the oxygenate compounds from the adsorbent with the recycled isobutane stream to form an isobutane stream with desorbed oxygenate compounds.

5. The process of claim 4 further comprising burning the isobutane stream with desorbed oxygenate compounds as fuel.

6. The process of claim 4 wherein the effluent stream from the etherification zone further comprises dimethyl ether, further comprising introducing the isobutane stream with desorbed oxygenate compounds into the depropanizer; and wherein the overhead vapor stream from the depropanizer further comprises the dimethyl ether.

7. The process of claim 1 wherein separating the liquid effluent stream in the depropanizer further comprises removing an upper side cut stream comprising isobutane, wherein the depropanizer stream has the concentration of isobutene of at least about 45%, and further comprising:
introducing the upper side cut stream into the etherification reaction zone.

8. The process of claim 1 wherein the depropanizer stream is a lower side cut stream, wherein the lower side cut stream has the isobutene concentration of at least about 45%, and wherein separating the liquid effluent stream in the depropanizer further comprises:
removing a bottoms stream comprising butene-2 and C₅₊ hydrocarbons.

9. The process of claim 8 wherein the oxygenate removal zone comprises an adsorbent, and further comprising:
introducing the overhead vapor stream from the depropanizer into the oxygenate removal zone;
desorbing the oxygenate compounds from the adsorbent with propane from the overhead vapor stream to form a propane stream with desorbed oxygenate compounds.

10. The process of claim 9 further comprising burning the propane stream with the desorbed oxygenate compounds as fuel.

11. The process of claim 1 further comprising removing sulfur from the feed prior to dehydrogenating the feed.

12. The process of claim 1 wherein the gaseous effluent stream from the dehydrogenation zone comprises hydrogen and methane, and further comprising:
separating the gaseous effluent stream into a hydrogen stream and a methane stream.

13. The process of claim 12 further comprising introducing at least a portion of the hydrogen stream to the saturation zone.

14. The process of claim 12 further comprising introducing at least a portion of the hydrogen stream to the dehydrogenation zone.

15. The process of claim 12 further comprising recovering at least a portion of the hydrogen stream.

16. The process of claim 1 further comprising introducing at least a portion of the isobutane stream from the saturation zone into the dehydrogenation reaction zone.

17. The process of claim 1 wherein the conversion of isobutene to isoprene is less than 90%.

18. The process of claim 1 wherein removing oxygenated compounds from the effluent stream comprises distilling the effluent stream.
